(19) United States
(12) Patent Application Publication
(10) Pub. No.: US 2003/0104007 A1
(43) Pub. Date: Jun. 5, 2003

(54) Pseudopterosin compounds of Symbiodinium spp isolated from Pseudopterogorgia elisabethae

(76) Inventors: Robert S. Jacobs, Santa Barbara, CA (US); Laura Mydlarz, Santa Barbara, CA (US); Russell G. Kerr, Boca Raton, FL (US)

Correspondence Address: Suzannah K. Sundby, Esq., Jacobson Holman PLLC, The Jenifer Building, 400 Seventh Street, N.W., Washington, DC, 20004, US

(21) Appl. No.: 10/264,026

(22) Filed: Oct. 4, 2002

Related U.S. Application Data
(60) Provisional application No. 60/327,028, filed on Oct. 5, 2001.
(60) Provisional application No. 60/340,833, filed on Dec. 19, 2001.

(51) Int. Cl. A61K 35/80; A61K 31/7056; C07H 19/00

(52) U.S. Cl. 424/195.17; 514/43; 536/27.2

(57) ABSTRACT

Disclosed herein are pseudopterosin compounds obtained from Symbiodinium spp. symbionts. Also disclosed are methods of obtaining, isolating, purifying or preparing at least one pseudopterosin compound comprising obtaining, isolating, purifying or preparing the pseudopterosin compound from at least one Symbiodinium spp. symbiont. In preferred embodiments, the host is Pseudopterogorgia, preferably, P. elisabethae. As disclosed, preferred pseudopterosin compounds and pseudopterosin compositions are of non-animal origin, substantially free of animal impurities, or both. Treatment methods using the pseudopterosin compounds and compositions are also disclosed.

### CROSS REFERENCE TO RELATED APPLICATIONS

This application claims the benefit of U.S. Provisional Patent Application Nos. 60/327,028, filed Oct. 5, 2001, and 60/340,833, filed Dec. 19, 2001, listing Robert S. Jacobs, Laura Mydlarz, and Russell G. Kerr as joint inventors, both of which are herein incorporated by reference in their entirety.

### BACKGROUND OF THE INVENTION

1. Field of the Invention

The present invention generally relates to pseudopterosin compounds isolated from Symbiodinium spp. symbionts and methods of making, isolating, and using thereof.

2. Description of the Related Art

Gorgonians (O. Gorgonacea, Ph. Cnidaria) are a diverse group of marine animals which are commonly known as sea feathers, sea whips and sea fans. Many species of gorgonians are found in abundance in the shallow-water reefs of the tropical Atlantic including regions of the Caribbean Sea. A few of the Caribbean gorgonians have been analyzed for their chemical content and found to be a source of many diverse organic substances such as steroids, prostaglandins, lactones, sesquiterpenoid derivatives and diterpenoid metabolites. Some of these substances have been found to be biologically active. In fact, pseudopterosins, seco-pseudopterosins, diterpene aglycones, and tricyclic diterpenes isolated from extracts of Pseudopterogorgia elisabethae exhibit anti-inflammatory and anti-proliferative activities. There are in excess of fifteen such pseudopterosin compounds that have been isolated and characterized in extracts of P. elisabethae. See Look, S. A, et al. (1986) J. Organic Chem. 51:5140-5145; Look, S. A, et al. (1986) PNAS 83:6238-6240; Look, S. A, et al. (1986) Tetrahedron 43:3363-3370; and Roussis, V., et al. (1990) J. Organic Chem. 55:4922-4925.

Unfortunately, in order to obtain these therapeutic compounds, the marine animals are sacrificed. As animal products are often undesirable for use in pharmaceutical and cosmetics, many have attempted to chemically synthesize these complex compounds. Others have attempted elucidate the biosynthetic pathway to make pseudopterosins with in vitro and in vivo recombinant systems that use crude or semi-purified enzyme extracts comprising terpene cyclase enzymes. See Kerr, R. G. et al., (1999) Organic Letters 1:2173-2175. These chemical and biosynthetic methods are expensive and often unsuccessful.

Thus, a need exists for pseudopterosin compounds of non-animal origin and inexpensive methods for obtaining the pseudopterosin compounds.

### SUMMARY OF THE INVENTION

The present invention relates to pseudopterosin compounds obtained from non-animal sources and methods of making and using thereof.

In some embodiments, the present invention relates to a method of obtaining, isolating, purifying or preparing at least one pseudopterosin compound comprising obtaining, isolating, purifying or preparing the pseudopterosin compound from at least one Symbiodinium spp. symbiont. In preferred embodiments, the Symbiodinium spp. symbiont belongs to phylotype B1. In some embodiments, the Symbiodinium spp. symbiont may be a cultured or cultivated cell line. In other embodiments, the Symbiodinium spp. symbiont may be obtained from a host such as Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Plexaura, Pocillopora, Pseudopterogorgia, Rhodactis, Stylophora, Tridacna, Zoanthus, and the like. In preferred embodiments, the host is Pseudopterogorgia, preferably, P. elisabethae.

The pseudopterosin compound is selected from the group consisting of pseudopterosins, seco-pseudopterosins, diterpene aglycones, tricyclic diterpenes, and derivatives thereof. The pseudopterosin compound may be a naturally occurring compound or a synthetic compound. In some embodiments, the pseudopterosin compound is Pseudopterosin A, Pseudopterosin B, Pseudopterosin C, Pseudopterosin D, Pseudopterosin E, Pseudopterosin F, Pseudopterosin G, Pseudopterosin H, Pseudopterosin I, Pseudopterosin J, Pseudopterosin K, Pseudopterosin L, Seco-Pseudopterosin A, Seco-Pseudopterosin B, Seco-Pseudopterosin C, Seco-Pseudopterosin D, Seco-Pseudopterosin E, or elisabethatriene. In preferred embodiments, the pseudopterosin compound is Pseudopterosin A, Pseudopterosin B, Pseudopterosin C, Pseudopterosin D, or elisabethatriene.

In some embodiments, the method further comprises culturing the Symbiodinium spp. symbiont. In some embodiments, the method further comprises incubating the Symbiodinium spp. symbiont with NaHCO₃.

In some embodiments, the present invention relates to pseudopterosin compounds and pseudopterosin compositions obtained from at least one Symbiodinium spp. symbiont. In preferred embodiments, the pseudopterosin compound or pseudopterosin composition is substantially free of animal impurities. In preferred embodiments, the pseudopterosin compound or pseudopterosin composition is of non-animal origin.

In some embodiments, the pseudopterosin compound is a glycoside. In these embodiments, the glycoside side chain may be modified.

In some embodiments, the present invention relates to a pharmaceutical composition comprising at least one pseudopterosin compound or at least one pseudopterosin composition obtained from at least one Symbiodinium spp. symbiont in a therapeutically effective amount and a pharmaceutically acceptable carrier. In some embodiments, the present invention relates to a cosmetic composition comprising at least one pseudopterosin compound or at least one pseudopterosin composition obtained from at least one Symbiodinium spp. symbiont in a therapeutically effective amount and a cosmetically acceptable carrier.

In some embodiments, the present invention relates to a method for treating, preventing, or inhibiting an infection, disease, or disorder related to an organism belonging to the kingdom Protista in a subject comprising administering to the subject a therapeutically effective amount of at least one pseudopterosin compound. The pseudopterosin compound may be a naturally occurring compound or a synthetic compound. The organism may be a flagellate, a ciliate, an opalinidae, or a sporozoan such as a plasmodium, a trypanosome, tetrahymenium, or a paramecium. In some embodiments, the organism is trichinosis, trypanosomiasis, leishmania, filariasis, or dracunculiasis. The infection, disease, or disorder may be malaria, Chagas' disease, African sleeping sickness, Leishmaniasis, giardiasis, or amebic dysentery.

In some embodiments, the present invention relates to a method of treating, preventing, or inhibiting a disease or disorder associated with inflammation, cell proliferation, pain, or a combination thereof in a subject comprising administering to the subject a therapeutically effective amount of at least one pseudopterosin compound or at least one pseudopterosin composition obtained from at least one Symbiodinium spp. symbiont.

In some embodiments, the present invention relates to an extract comprising at least one pseudopterosin compound in an amount that is greater than amounts obtained from coral extracts. The extract may be an algal extract. The extract may exhibit a greater pseudopterosin compound activity per gram of extract as compared to coral extracts.

In some embodiments, the present invention relates to kits comprising the pseudopterosin compounds and pseudopterosin compositions and instructions for use. The kits may further include supplementary active compounds, wound dressings, applicators for administration, or combinations thereof.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention and together with the description serve to explain the principles of the invention.

### DESCRIPTION OF THE DRAWINGS

This invention is further understood by reference to the drawings wherein:

FIG. 1 is a thin layer chromatogram of Symbiodinum extracts performed in the field.

FIG. 2 is a thin layer chromatogram of Symbiodinium extracts performed in the laboratory.

FIG. 3 is a HPLC chromatogram of the chloroform fraction of a standard for Pseudopterosins A-D.

FIG. 4 is a HPLC chromatogram of the chloroform fraction from S. microandriaticum.

FIG. 5 is an HPLC chromatogram of PsA standard and radioactivity of PsA from ³H GGPP incubation.

FIG. 6 is an HPLC chromatogram of PsC standard and radioactivity of PsC from ³H GGPP incubation.

### DETAILED DESCRIPTION OF THE INVENTION

Recently, the present inventors have hypothesized that symbionts of P. elisabethae may be involved in the production of pseudopterosin compounds. Until the present invention, however, it was unknown whether such symbionts, if involved, are involved in only a part of the synthetic pathway so that neither the symbiont nor the host can produce pseudopterosin compounds alone.

As disclosed herein, a Symbiodinum spp. symbiont, is involved in the synthesis of pseudopterosin compounds and can produce pseudopterosin compounds without the aid of the host, P. elisabethae. Specifically, as shown in the detailed examples below, crude Symbiodinum spp. symbiont preparations freshly isolated from live P. elisabethae incubated with ¹⁴C labeled sodium bicarbonate provides radiolabeled PsA, PsC, and elisabethatriene. The radiolabeled compounds suggest that the symbiont fixes and incorporates CO₂ into the pseudopterosin biosynthetic pathway. Additionally, organic extracts of purified Symbiodinum spp. symbiont preparations showed endogenous levels of PsA, PsB, PsC, PsD and elisabethatriene and cell free extracts of purified Symbiodinum spp. symbiont prepared from flash frozen P. elisabethae in phosphate buffer were able to incorporate ³H labeled geranylgeranyl pyrophosphate into labeled PsA and PsC. Thus, pseudopterosin compounds may be produced by or isolated from Symbiodinum symbionts.

As used herein, “pseudopterosin compounds” include natural and synthetic pseudopterosins, seco-pseudopterosins, diterpene aglycones, and tricyclic diterpenes that may be produced by, synthesized in, or isolated from species belonging to the genus Pseudopterogorgia, Symbiodinum spp. symbionts, or derivatives thereof such as Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Seco-Pseudopterosin A (SPsA), Seco-Pseudopterosin B (SPsB), Seco-Pseudopterosin C (SPsC), Seco-Pseudopterosin D (SPsD), Seco-Pseudopterosin E (SPsE), and Elisabethatriene. As used herein, “pseudopterosin compositions” include cellular extracts of Symbiodinum spp. symbionts or hosts having pseudopterosin compounds.

Derivatives of pseudopterosin compounds include compounds that have chemical structures and activities that are similar to those compounds produced by, synthesized in, or isolated from Symbiodinum spp. symbionts or hosts thereof. Derivatives of pseudopterosin compounds may be synthesized by derivatizing the various naturally occurring pseudopterosins and seco-pseudopterosins which are isolated from Symbiodinum hosts, such as sea whips, according to known procedures such as those described by Look et al. (1986) PNAS 83:6238-6240; Look et al. (1986) J. Org. Chem. 51:5140-5145; Look et al. (1987) Tetrahedron 43:3363-3370; Roussis et al. (1990) J. Org. Chem. 55:4916-4922; and U.S. Pat. Nos. 4,849,410, 4,745,104, and 5,624,911, which are herein incorporated by reference.

As used herein, “substantially free of animal impurities” means that less than 10%, preferably less than 5%, more preferably less than 1% is animal protein or cellular debris. Compounds and compositions that are substantially free of animal impurities are preferably obtained from sources of non-animal origin.

Although the Examples below exemplify the isolation and characterization of PsA, PsB, PsC, PsD, and elisabethatriene, from Symbiodinium spp. symbionts of P. elisabethae, one of ordinary skill in the art should be readily able to obtain a variety of other pseudopterosin compounds from Symbiodinium spp. symbionts of other hosts by following the methods disclosed herein without undue experimentation. For example, according to the present invention, pseudopterosin compounds may be obtained from Symbiodinium spp. isolated from hosts such as Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Plexaura, Pocillopora, Pseudoterogorgia, Rhodactis, Stylophora, Tridacna, Zoanthus, and the like. Examples of specific Symbiodinium spp. symbionts include S. kawagutii, S. goreaui, S. muscatinei, S. pulchrorum, S. bermudense, S. californium, S. microadriatiucum, S. pilosum, S. meandrinae, S. corculorum, S. linucheae, and the like. Preferred Symbiodinium spp. belong to phylotype B1 as classified by LaJeunesse, J. Phycol. (2001) 37:866-880, which is herein incorporated by reference.

Additionally, various pseudopterosin compounds may be obtained from symbionts isolated from P. elisabethae found in different geographical locations as different P. elisabethae populations in the Bahamas produce different pseudopterosin compounds. For example, PsA through PsD, were originally found in P. elisabethae populations off Crooked Island in the Bahamas. See Clardy, J. et al. (1986) J. Org. Chem. 51:5140-5145, which is herein incorporated by reference. PsE through PsJ were found in P. elisabethae populations in Bermuda and PsK through PsL were found in populations off Great Abaco Island. See Fenical, W. et al. (1990) J. Org. Chem. 55(16):4916, which is herein incorporated by reference.

This distribution of the various pseudopterosin compounds is likely the result of different environmental conditions of the areas in which the hosts and symbionts are located. Thus, it is likely that one pseudopterosin compound may be obtained from one symbiont in a particular area and a second pseudopterosin compound may be obtained from a symbiont in a second area. The symbionts may be the same or different. Thus, it is contemplated that different pseudopterosin compounds from a variety of symbionts and hosts may be obtained according to the present invention.

As disclosed in the Examples herein, pseudopterosin compounds may be produced by or isolated from Symbiodinium spp. symbionts. The pseudopterosin compounds may be obtained from freshly isolated symbionts. Alternatively, the pseudopterosin compounds may be obtained from cultured or cultivated symbionts such as those from established cultures and cell lines. Cell cultures and cell lines may be made by conventional methods known in the art. See, e.g. LaJeunesse (2001) and Trench, R. K. et al. (2000) J. Exp. Mar. Biol. Ecol. 249:219-233, which are herein incorporated by reference. Thus, the present invention provides pseudopterosin compounds of non-animal origin and methods of producing the pseudopterosin compounds.

The structural formulas of a few of these pseudopterosin compounds are as follows:

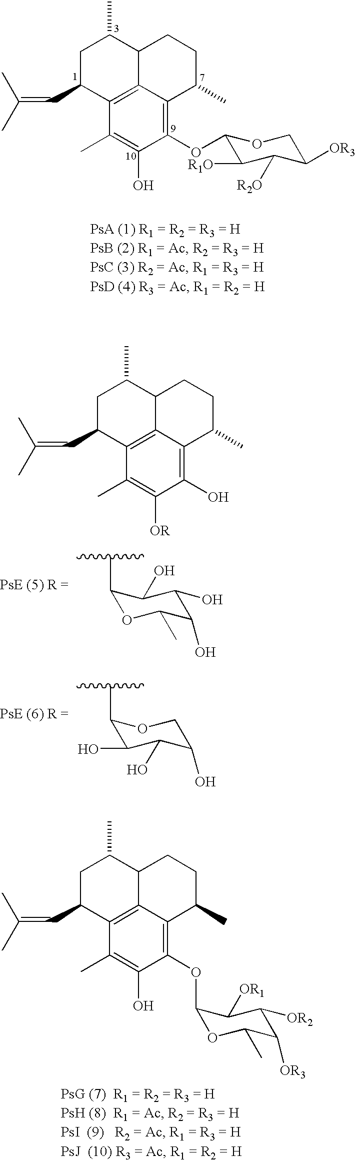

As provided in Scheme 1 below, elisabethatriene (18) is the cyclase product that is a precursor for a variety of pseudopterosin compounds in the biosynthesis of pseudopterosin compounds. Thus, as disclosed herein, the present invention provides elisabethatriene and pseudopterosin compounds having elisabethatriene as a precursor of a non-animal origin and methods of producing thereof. In preferred embodiments, the present invention provides pseudopterosin compounds produced by or isolated from symbionts, such as Symbiodinium spp. symbionts. In some embodiments, the host of the symbiont is Pseudopterogorgia, such as P. elisabethae.

These pseudopterosin compounds may be used as the active ingredient in pharmaceuticals and cosmetics. Alternatively, the pseudopterosin compounds may be used as prodrugs that after administration to a subject are converted in vivo to other pseudopterosins and seco-pseudopterosins such as PsM (19) and elisabethadiol (20).

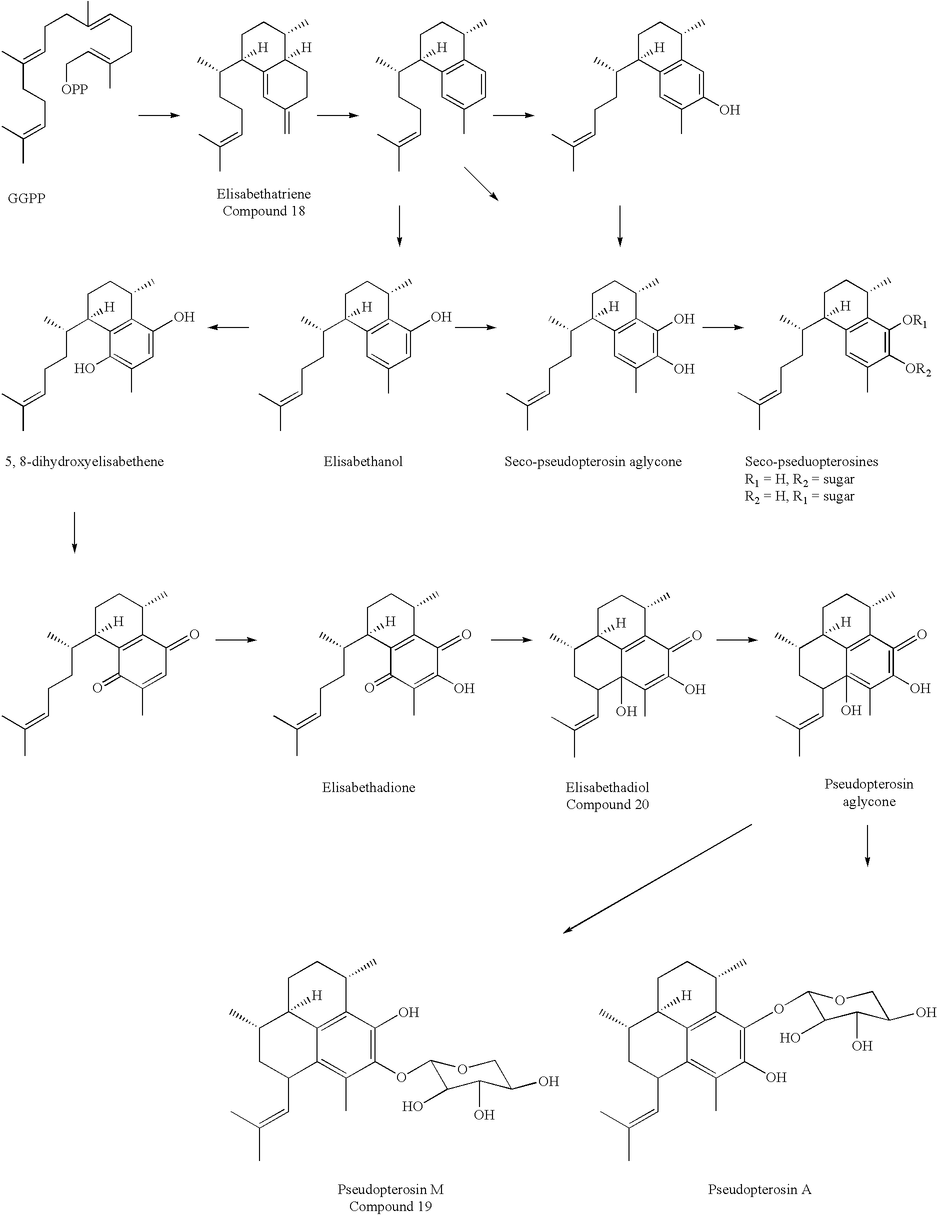

The terms and abbreviations used in the instant disclosure have their normal meanings unless otherwise designated.

As used in the present application, the following definitions apply:

In accordance with a convention used in the art,

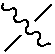

is used in structural formulas herein to depict the bond that is the point of attachment of the moiety or substituent to the core or backbone structure.

Where chiral carbons are included in chemical structures, unless a particular orientation is depicted, both sterioisomeric forms are intended to be encompassed.

An “alkyl” is intended to mean a straight or branched chain monovalent radical of saturated and/or unsaturated carbon atoms and hydrogen atoms, such as methyl (Me), ethyl (Et), propyl (Pr), isopropyl (i-Pr), butyl (Bu), isobutyl (i-Bu), t-butyl (t-Bu), ethenyl, pentenyl, butenyl, propenyl, ethynyl, butynyl, propynyl, pentynyl, hexynyl, and the like, which may be unsubstituted (i.e., contain only carbon and hydrogen) or substituted by one or more suitable sustituents as defined below (e.g., one or more halogen, such as F, Cl, Br, or I, with F and Cl being preferred). A “lower alkyl group” is intended to mean an alkyl group having from 1 to 8 carbon atoms in its chain.

A “cycloalkyl” is intended to mean a non-aromatic monovalent monocyclic, bicyclic, or tricyclic radical containing 3-14 carbon ring atoms, each of which may be saturated or unsaturated, and which may be unsubstituted or substituted by one or more suitable substituents as defined below, and to which may be fused one or more heterocycloalkyl groups, aryl groups, or heteroaryl groups, which themselves may be unsubstituted or substituted by one or more substituents. Illustrative examples of cycloalkyl groups include following moieties:

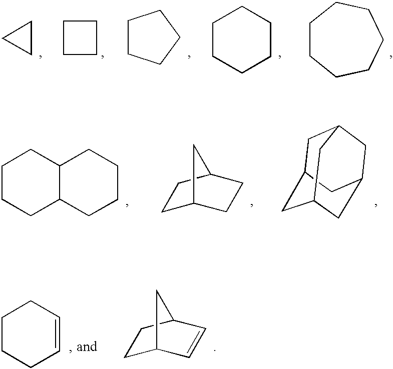

A “heterocycloalkyl” is intended to mean a non-aromatic monovalent monocyclic, bicyclic, or tricyclic radical, which is saturated or unsaturated, containing 3-18 ring members, which includes 1-5 heteroatoms selected from nitrogen, oxygen, and sulfur, where the radical is unsubstituted or substituted by one or more suitable substituents as defined below, and to which may be fused one or more cycloalkyl groups, aryl groups, or heteroaryl groups, which themselves may be unsubstituted or substituted by one or more suitable substituents. Illustrative examples of heterocycloalkyl groups include the following moieties:

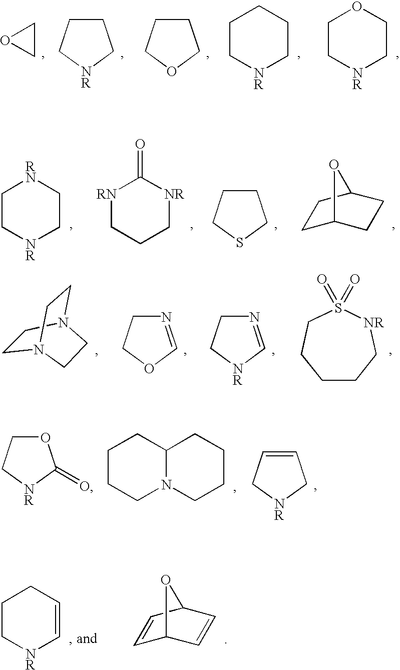

An “aryl” is intended to mean an aromatic monovalent monocyclic, bicyclic, or tricyclic radical containing 6, 10, 14, or 18 carbon ring members, which may be unsubstituted or substituted by one or more suitable substituents as defined below, and to which may be fused one or more cycloalkyl groups, heterocycloalkyl groups, or heteroaryl groups, which themselves may be unsubstituted or substituted by one or more suitable substituents. Thus, the term “aryl group” includes a benzyl group (Bzl). Illustrative examples of aryl groups include the following moieties:

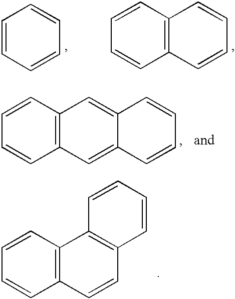

A “heteroaryl” is intended to mean an aromatic monovalent monocyclic, bicyclic, or tricyclic radical containing 4-18 ring members, including 1-5 heteroatoms selected from nitrogen, oxygen, and sulfur, which may be unsubstituted or substituted by one or more suitable substituents as defined below, and to which may be fused one or more cycloalkyl groups, heterocycloalkyl groups, or aryl groups, which themselves may be unsubstituted or substituted by one or more suitable substituents. Illustrative examples of heteroaryl groups include the following moieties:

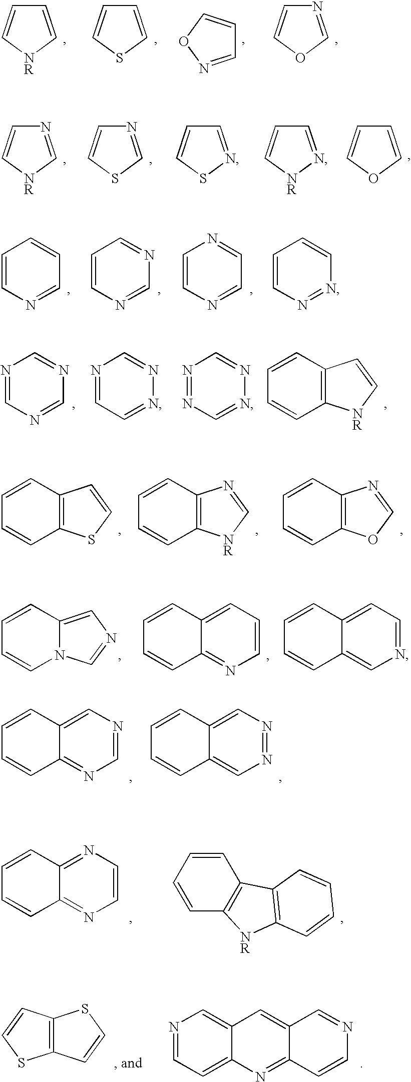

A “heterocycle” is intended to mean a heteroaryl or heterocycloalkyl group (each of which, as defined above, are optionally substituted).

An “acyl” is intended to mean a —C(O)—Rₐ radical, where Rₐ is a suitable substituent as defined below.

A “thioacyl” is intended to mean a —C(S)—Rₐ radical, where Rₐ is a suitable substituent as defined below.

A “sulfonyl” is intended to mean a —SO₂Rₐ radical, where Rₐ is a suitable substituent as defined below.

A “hydroxy” is intended to mean the radical —OH.

An “amino” is intended to mean the radical —NH₂.

An “alkylamino” is intended to mean the radical —NHRₐ, where Rₐ is an alkyl group.

A “dialkylamino” is intended to mean the radical —NRₐR_{b}, where Rₐ and R_{b} are each independently an alkyl group.

An “alkoxy” is intended to mean the radical —ORₐ, where Rₐ is an alkyl group. Exemplary alkoxy groups include methoxy, ethoxy, propoxy, and the like.

An “alkoxycarbonyl” is intended to mean the radical —C(O)ORₐ, where Rₐ is an alkyl group.

An “alkylsulfonyl” is intended to mean the radical —SO₂Rₐ, where Rₐ is an alkyl group.

An “alkylaminocarbonyl” is intended to mean the radical —C(O)NHRₐ, where Rₐ is an alkyl group.

A “dialkylaminocarbonyl” is intended to mean the radical —C(O)NRₐR_{b}, where Rₐ and R_{b} are each independently an alkyl group.

A “mercapto” is intended to mean the radical —SH.

An “alkylthio” is intended to mean the radical —SRₐ, where Rₐ is an alkyl group.

A “carboxy” is intended to mean the radical —C(O)OH.

A “carbamoyl” is intended to mean the radical —C(O)NH₂.

An “aryloxy” is intended to mean the radical —OR_{c}, where R_{c} is an aryl group.

A “heteroaryloxy” is intended to mean the radical —OR_{d}, where R_{d} is a heteroaryl group.

An “arylthio” is intended to mean the radical —SR_{c}, where R_{c} is an aryl group.

A “heteroarylthio” is intended to mean the radical —SR_{d}, where R_{d} is a heteroaryl group.

A “leaving group” (Lv) is intended to mean any suitable group that will be displaced by a substitution reaction. One of ordinary skill in the art will know that any conjugate base of a strong acid can act as a leaving group. Illustrative examples of suitable leaving groups include, but are not limited to, —F, —Cl, —Br, alkyl chlorides, alkyl bromides, alkyl iodides, alkyl sulfonates, alkyl benzenesulfonates, alkyl p-toluenesulfonates, alkyl methanesulfonates, triflate, and any groups having a bisulfate, methyl sulfate, or sulfonate ion.

A “protecting group” is intended to refer to groups that protect one or more inherent functional group from premature reaction. Suitable protecting groups may be routinely selected by those skilled in the art in light of the functionality and particular chemistry used to construct the compound. Examples of suitable protecting groups are described, for example, in Greene and Wutz, Protecting Groups in Organic Synthesis, 2^{nd} edition, John Wiley and Sons, New York, N.Y. (1991).

The term “suitable organic moiety” is intended to mean any organic moiety recognizable, such as by routine testing, to those skilled in the art as not adversely affecting the inhibitory activity of the inventive compounds. Illustrative examples of suitable organic moieties include, but are not limited to, hydroxyl groups, alkyl groups, oxo groups, cycloalkyl groups, heterocycloalkyl groups, aryl groups, heteroaryl groups, acyl groups, sulfonyl groups, mercapto groups, alkylthio groups, alkoxy groups, carboxy groups, amino groups, alkylamino groups, dialkylamino groups, carbamoyl groups, arylthio groups, heteroarylthio groups, and the like.

The term “substituent” or “suitable substituent” is intended to mean any suitable substituent that may be recognized or selected, such as through routine testing, by those skilled in the art. Illustrative examples of suitable substituents include hydroxy groups, halogens, oxo groups, alkyl groups, acyl groups, sulfonyl groups, mercapto groups, alkylthio groups, alkyloxy groups, cycloalkyl groups, heterocycloalkyl groups, aryl groups, heteroaryl groups, carboxy groups, amino groups, alkylamino groups, dialkylamino groups, carbamoyl groups, aryloxy groups, heteroaryloxy groups, arylthio groups, heteroarylthio groups, and the like.

The pseudopterosin compounds are known to additionally to exist as glycosides, see for example, Scheme 1, and are represented as o-glycosides. Glycosides can include simple phenolic compounds, tannins, courmarins, anthraquinones, nathoquinones, flavones, and other biosynthetic natural products. The anti-inflammatory glycosides of salicylic acid are widely distributed in higher plants. The polarity and pharmacokinetic properties of steroid and terpene glycosides are well known to be altered by the glycoside side chain. Absorption and distribution of a drug is dependent on its lipid solubility which in turn is directly proportional to non-specific binding to plasma proteins. For example, the longer the sugar side chain, the more polar the drug and hence its protein binding is altered. Certain intermediates in the biosynthesis of the pseudopterosin compounds may be glycosides. Such glycosides and other pseudopterosin compounds may be optimized by altering the sugar side chain, the polarity, the protein binding activity, or a combination thereof. These optimized compounds are useful for formulation of dosages and therapeutic maintenance and are within the scope of the present invention.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

It is understood that while a pseudopterosin compound herein may exhibit the phenomenon of tautomerism, the structural formulas within this specification expressly depict only one of the possible tautomeric forms. It is therefore to be understood that the structural formulas herein are intended to represent any tautomeric form of the depicted compound and is not to be limited merely to a specific compound form depicted by the structural formulas. It is also understood that the structural formulas are intended to represent any configurational form of the depicted compound and is not to be limited merely to a specific compound form depicted by the structural formulas.

Some of the pseudopterosin compounds may exist as single stereoisomers (i.e., essentially free of other stereoisomers), racemates, or mixtures of enantiomers, diastereomers, or both. All such single stereoisomers, racemates and mixtures thereof are intended to be within the scope of the present invention. Preferably, the pseudopterosin compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, if the compounds of the present invention are made synthetically, they are used in a form that is at least 90% optically pure, that is, a form that contains at least 90% of a single isomer (80% enantiomeric excess (e.e.) or diastereomeric excess (d.e.), more preferably at least 95% (90% e.e. or d.e.), even more preferably at least 97.5% (95% e.e. or d.e.), and most preferably at least 99% (98% e.e. or d.e.).

Additionally, the pseudopterosin compounds may be in a solvated or unsolvated form. A “solvate” is intended to mean a pharmaceutically acceptable solvate form of a specified compound that retains the biological effectiveness of such compound. Examples of solvates include at least one pseudopterosin compound in combination with water, isopropanol, ethanol, methanol, dimethyl sulfoxide, ethyl acetate, acetic acid, ethanolamine, or acetone. Also included are miscible formulations of solvate mixtures such as a pseudopterosin compound in combination with an acetone and ethanol mixture. In a preferred embodiment, the solvate includes a pseudopterosin compound in combination with about 20% ethanol and about 80% acetone. Thus, the pseudopterosin compounds of the present invention include the hydrated as well as the non-hydrated forms.

As indicated above, the pseudopterosin compounds of the present invention also include active tautomeric and stereoisomeric forms of the structural formulas herein which may be readily obtained using techniques known in the art. For example, optically active (R) and (S) isomers may be prepared via a stereospecific synthesis, e.g., using chiral synthons and chiral reagents, or racemic mixtures may be resolved using conventional techniques.

Additionally, the compounds of the invention include pharmaceutically acceptable salts, multimeric forms, prodrugs, active metabolites, precursors and salts of such metabolites of pseudopterosin compounds.

The term “pharmaceutically acceptable salts” refers to salt forms that are pharmacologically acceptable and substantially non-toxic to the subject being treated with the compound of the invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from organic bases such as amines, benzylamines, piperidines, and pyrrolidines.

The term “multimer” refers to multivalent or multimeric forms of active forms of the pseudopterosin compounds of the invention. Such “multimers” may be made by linking or placing multiple copies of an active compound in close proximity to each other, e.g., using a scaffolding provided by a carrier moiety. Multimers of various dimensions (i.e., bearing varying numbers of copies of an active compound) may be tested to arrive at a multimer of optimum size with respect to receptor binding. Provision of such multivalent forms of active receptor-binding compounds with optimal spacing between the receptor-binding moieties may enhance receptor binding (see, for example, Lee et al., Biochem., 1984, 23:4255). The artisan may control the multivalency and spacing by selection of a suitable carrier moiety or linker units. Useful moieties include molecular supports containing a multiplicity of functional groups that can be reacted with functional groups associated with the active compounds of the invention. A variety of carrier moieties may be used to build highly active multimers, including proteins such as BSA (bovine serum albumin) or HSA, peptides such as pentapeptides, decapeptides, pentadecapeptides, and the like, as well as non-biological compounds selected for their beneficial effects on absorbability, transport, and persistence within the target organism. Functional groups on the carrier moiety, such as amino, sulfhydryl, hydroxyl, and alkylamino groups, may be selected to obtain stable linkages to the compounds of the invention, optimal spacing between the immobilized compounds, and optimal biological properties.

“A pharmaceutically acceptable prodrug” is a compound that may be converted under physiological conditions or by solvolysis to the specified compound or to a pharmaceutically acceptable salt of such compound. “A pharmaceutically active metabolite” is intended to mean a pharmacologically active product produced through metabolism in the body of a specified compound or salt thereof. Prodrugs and active metabolites of a compound may be identified using routine techniques known in the art. See, e.g., Bertolini, G. et al., J. Med. Chem., 40, 2011-2016 (1997); Shan, D. et al., J. Pharm. Sci., 86 (7), 765-767; Bagshawe K., Drug Dev. Res., 34, 220-230 (1995); Bodor, N., Advances in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

If the pseudopterosin compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyrvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the pseudopterosin compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds, agents and salts may exist in different crystal or polymorphic forms, all of which are intended to be within the scope of the present invention and specified structural formulas.

By substantially following the procedures described herein, one skilled in the art can prepare other pseudopterosin compounds that fall within the scope of the present invention.

Since various pseudopterosin compounds are known to be anti-inflammatory agents, anti-proliferative agents, analgesic agents, or a combination thereof, the present invention also provides methods of treating diseases and disorders related to or associated with inflammation, abnormal cell proliferation, pain, or a combination thereof in a subject comprising administering at least one pseudopterosin compound of the present invention to the subject. As used herein “pseudopterosin compound activity” includes anti-inflammatory activity, anti-proliferative activity, and analgesic activity.

The pseudopterosin compounds of the present invention may be used to treat, prevent, or inhibit rheumatoid arthritis, osteoarthritis, rheumatic carditis, collagen and auto-immune diseases such as myasthenia gravis, allergic diseases, bronchial asthma and ocular and skin inflammatory diseases such as poison ivy. The pseudopterosin compounds of the present invention may be used to treat, prevent, or inhibit proliferative diseases such as psoriasis.

The pseudopterosin compounds of the present invention are also useful as adjuvant therapy associated with organ and tissue transplants and any neurological disease involving the metabolism of nervous tissue phospholipid such as multiple sclerosis. Because of their selective antagonism of chemical irritation (i.e., PMA inflammation) the compounds can be useful in the treatment of insect bites, bee or wasp stings or any venom in which a major constituent is the enzyme phospholipase A₂.

As some pseudopterosin compounds are potent non-narcotic analgesics, the pseudopterosin compounds of the present invention may be used to alleviate pain resulting from traumatic injury or acute progressive disease, such as post-operative pain, burns, or other conditions involving a coincident inflammation.

The pseudopterosin compounds of the present invention may also be used for treating lesions related to chemotherapy and radiation which include ulceration of the skin, oral cavity, trachea, bronchi, digestive tract and colon. The compounds may also be used for treating inflammatory conditions of the eye, ulceration of the nasal passage, and anaphylactic shock related to treatments for radiation, burns, or both.

Additionally, as shown in Example 6, pseudopterosin compounds affect the activity of protists such as those belonging to Tetrahymena. Thus, pseudopterosin compounds may be used for treating, preventing, or inhibiting an infection, disease, or disorder related to an organism belonging to the kingdom Protista in a subject comprising administering to the subject a therapeutically effective amount of at least one pseudopterosin compound. The organism may be a flagellate, a ciliate, an opalinidae, or a sporozoan such as a plasmodium, a trypanosome, tetrahymenium, or a paramecium. Examples of such infections, diseases, and disorders include malaria, Chagas' disease, African sleeping sickness, Leishmaniasis, giardiasis, or amebic dysentery.

The pseudopterosin compounds of the present invention may be used in combination with or as a substitution for treatments of the above conditions. For example, the compounds of the invention may be used alone or in combination with morphine or other analgesics to treat pain and inflammation such as that resulting from surgical procedures. Other diseases, disorders, and conditions which may be treated with the compounds of the present invention include hypersensitivity pneumonitis, inflammation associated with coronary angioplasty, arthritis such as rheumatoid arthritis and osteoarthritis, nephritis, and conjunctivitis.

A compound of the present invention may be administered in a therapeutically effective amount to a mammal such as a human. A therapeutically effective amount may be readily determined by standard methods known in the art. As defined herein, a therapeutically effective amount of a compound of the invention ranges from about 0.1 to about 25.0 mg/kg body weight, preferably about 1.0 to about 20.0 mg/kg body weight, and more preferably about 10.0 to about 20.0 mg/kg body weight. Preferred topical concentrations include about 0.1% to about 20.0% in a formulated salve. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of the compound can include a single treatment or, preferably, can include a series of treatments.

In a preferred example, a subject is treated with a compound of the invention in the range of between about 0.1 to about 25.0 mg/kg body weight, at least one time per week for between about 5 to about 8 weeks, and preferably between about 1 to about 2 weeks. It will also be appreciated that the effective dosage of the compound used for treatment may increase or decrease over the course of a particular treatment. Changes in dosage may result and become apparent by standard diagnostic assays known in the art. In some conditions chronic administration may be required.

The pharmaceutical compositions of the invention may be prepared in a unit-dosage form appropriate for the desired mode of administration. The compositions of the present invention may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the condition to be treated, and the chosen active compound.

It will be appreciated that the actual dosages of the agents used in the compositions of this invention will vary according to the particular complex being used, the particular composition formulated, the mode of administration, and the particular site, host, and disease being treated. Optimal dosages for a given set of conditions may be ascertained by those skilled in the art using conventional dosage-determination tests in view of the experimental data for a given compound. Administration of prodrugs may be dosed at weight levels that are chemically equivalent to the weight levels of the fully active forms.

The pseudopterosin compounds of the present invention can be incorporated into pharmaceutical compositions suitable for administration. Pharmaceutical compositions of this invention comprise an therapeutically effective amount of at least one pseudopterosin compound of the present invention and an inert, pharmaceutically acceptable carrier or diluent. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Supplementary active compounds include other pseudopterosins and seco-pseudopterosins such as those described in U.S. Pat. Nos. 4,745,104, 4,849,410, and 5,624,911, all of which are herein incorporated by reference. Supplementary compounds also include hydrocortisone, cox inhibitors such as indomethacin or salicylates, fixed anesthetics such as lidocaine, opiates, and morphine.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, at least one pseudopterosin compound is dissolved in DMSO and diluted with water.

The composition may also be in the form of a solution of a salt form of the active ingredient in an appropriate aqueous vehicle such as water or isotonic saline or dextrose solution.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions suitable for injectable use include sterile aqueous solutions (where water soluble) or dispersions and sterile powders for the extemporaneous preparation of sterile injectable solutions or dispersion. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a therapeutically effective amount of a compound of the invention in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active compound plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, foams, powders, sprays, aerosols or creams as generally known in the art.

For example, for topical formulations, pharmaceutically acceptable excipients may comprise solvents, emollients, humectants, preservatives, emulsifiers, and pH agents. Suitable solvents include ethanol, acetone, glycols, polyurethanes, and others known in the art. Suitable emollients include petrolatum, mineral oil, propylene glycol dicaprylate, lower fatty acid esters, lower alkyl ethers of propylene glycol, cetyl alcohol, cetostearyl alcohol, stearyl alcohol, stearic acide, was, and others known in the art. Suitable humectants include glycerin, sorbitol, and others known in the art. Suitable emulsifiers include glyceryl monostearate, glyceryl monoleate, stearic acid, polyoxyethylene cetyl ether, polyoxyethylene cetostearyl ether, polyoxyethylene stearyl ether, polyethylene glycol stearate, and others known in the art. Suitable pH agents include hydrochloric acid, phosphoric acid, diethanolamine, triethanolamine, sodium hydroxide, monobasic sodium phosphate, dibasic sodium phosphate, and others known in the art. Suitable preservatives include benzyl alcohol, sodium benzoate, parabens, and others known in the art.

For administration to the eye, the compound of the invention is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmnaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g, containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the pseudopterosin compounds of the present invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

In some embodiments, the pseudopterosin compounds of the present invention may be prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pseudopterosin compounds and pseudopterosin compositions of the present invention may be provided in kits along with instructions for use. The kits may further include supplementary active compounds, wound dressings, applicators for administration, or combinations thereof.

The pseudopterosin compounds of the present invention may be prepared using the reaction routes and synthesis schemes as described herein, employing the techniques available in the art using starting materials that are readily available. The preparation of preferred pseudopterosin compounds of the present invention is described in detail in the following examples, but the artisan will recognize that the chemical reactions described may be readily adapted to prepare a number of other compounds falling within the scope of the present invention. For example, a variety of pseudopterosin compounds may be made by obtaining elisabethatriene from cultures of at least one Symbiodinium spp. symbiont and then chemically modifying elisabethatriene by conventional methods in the art. See e.g., Look et al. (1986) PNAS 83:6238-6240; Look et al. (1986) J. Org. Chem. 51:5140-5145; Look et al. (1987) Tetrahedron 43:3363-3370; Roussis et al. (1990) J. Org. Chem. 55:4916-4922; and U.S. Pat. Nos. 4,849,410, 4,745,104, and 5,624,911.

Occasionally, the reaction routes and synthesis schemes set forth herein may not be applicable to each compound included within the disclosed scope of the invention. The compounds for which this occurs will be readily recognized by those skilled in the art. In all such cases, either the reactions can be successfully performed by conventional modifications to the disclosed reactions routes and schemes. For example, one of ordinary skill in the art will be able to modify the disclosed reactions by the appropriate protection of interfering groups, by changing one or more of the reagents to other conventional reagents, or by routine modification of the reaction conditions. Alternatively, other reactions disclosed herein or otherwise known to one of ordinary skill in the art will be applicable to the preparation of the corresponding compounds of the invention.

The following examples are intended to illustrate but not to limit the invention.

### Example 1

### Extraction and Isolation

Samples of coral, P. elisabethae, were collected from Sweetings Cay, Bahamas at a depth of about 15 to about 30 meters in May 2001. Live coral was homogenized in a tissue homogenizer with filtered seawater and EDTA. The seawater was filtered with a 0.22 μm filter (Steritop™, vacuum filter 0.22 μm, Millipore). The homogenate was filtered through 4 layers of cheesecloth. The filtrate was centrifuged at 1000×g to yield an algal pellet. The pellet was rinsed with filtered seawater and centrifuged 10 times.

The symbionts associated with P. elisabethae collected from Sweetings Cay, Bahamas, were typed by Todd C. LaJeunesse as B1 Symbiodinium using denaturing gradient gel electrophoresis (DGGE) on the ITS2 region according to LaJeunesse (2001) which is herein incorporated by reference, and the ITS2 sequences of the symbionts were found to be identical to invalid S. pulchrorum and S. burmudense.

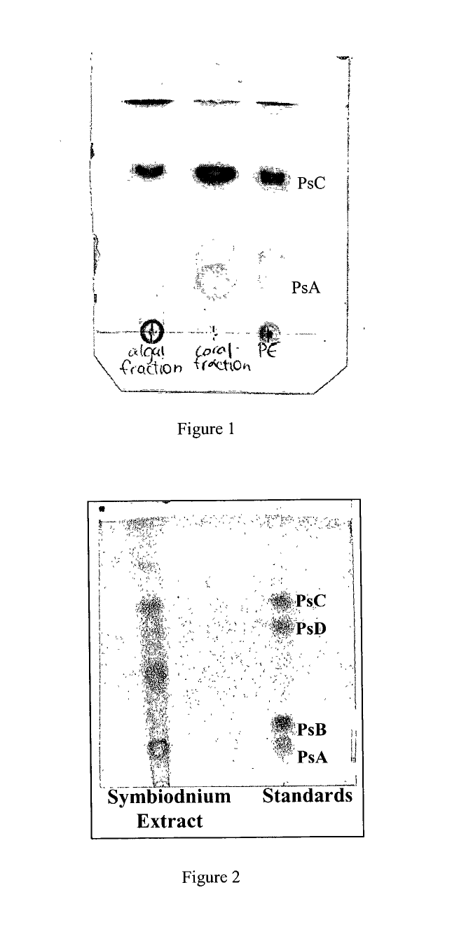

An organic extract in 100 ml chloroform was made of the algal pellet. Thin Layer Chromatography (TLC) analysis was conducted in the field using the algae pellet and standards to detect the presence of pseudopterosin compounds in the algae fraction. FIG. 1 shows that pseudopterosin compounds are present in the algae fraction from the TLC analysis conducted in the field. FIG. 2 shows the results of the a TLC analysis conducted in the lab which confirms the presence of endogenous concentrations of pseudopterosin compounds, PsA, PsB, PsC, and PsD, in the algal fraction. The standards used for pseudopterosins A-D were purified from P. elisabethae (collected at Sweetings Cay, Bahamas) and extracted by conventional chromatographic methods and structurally characterized by NMR spectroscopy.

### Example 2

### In Vivo Incubation with NaH¹⁴CO₃

The following in vivo experiment was repeated three times. An algae pellet prepared according to Example 1 was suspended in filtered seawater. The algae cells were diluted to about 4×10⁵ cells/ml in filtered seawater and 40 ml of cells were placed in a sterile erlenmeyer flask. To each, 0.5 μCi/ml ¹⁴C labeled sodium bicarbonate (NaH¹⁴CO₃) was added. The cells were incubated for 24 hours in the presence of a plant growth light. The cells were then harvested, concentrated by centrifugation and flash frozen in liquid nitrogen-for later analysis. In later experiments the algae pellet was prepared with optimized conditions, wherein 2×10⁶ cells/ml of cells were suspended in filtered seawater and 9 ml of cells were incubated with 2 μCi/ml of ¹⁴C labeled sodium bicarbonate (NaH¹⁴CO₃). These cells were incubated for 48 hours in the presence of a plant growth light.

The algal pellet from the above experiments was extracted using 100 ml HPLC grade chloroform. The crude extract was partitioned between hexanes and (9:1) methanol (MeOH)/water (100 ml total). The hexanes fraction was analyzed on reverse phase HPLC in 100% MeOH and elisabethatriene was purified and collected for radioisotope analysis with a liquid scintillation counter. The MeOH/water extract was run on straight phase HPLC on a gradient from 60% hexane/40% ethyl acetate to 100% ethyl acetate. Pseudopterosins A, B, C and D were purified by comparing the retention times to the retention times of the standards of Example 1 and collected for radioisotope analysis with a liquid scintillation counter. The elisabethatriene standard was obtained from the hexane fraction of a crude extract of P. elisabethae collected in the Florida Keys which was purified by normal phase HPLC (hexanes/ethyl acetate eluent) equipped with a diode array detector. The structure was elucidated using NMR and MS spectroscopic methods. See Kerr, R. G. et al., (2000) Tetrahedron 56:9569-9574, which is herein incorporated by reference.

Table 1 shows the radioactivity of each pseudopterosin compound and the intermediate elisabethatriene after purification by HPLC.

TABLE 1

| Compound | Radioactivity in DPM |
|---|---|
| Elisabethatriene | 1126.00 |
| PsA | 147.75 |
| PsB | 89.36 |
| PsC | 63.50 |
| PsD | 50.58 |

Table 2 provides the summarized results of all experiments (done in triplicate) which are normalized as DPM per 10⁶ cells.

TABLE 2

| Compound | DPM/10⁶ cells |
|---|---|
| Elisabethatriene | 5.323 ± 2.42 |
| PsA | 0.685 ± 0.239 |
| PsB | 0.498 ± 0.110 |
| PsC | 0.530 ± 0.122 |
| PsD | 0.476 ± 0.120 |

In the first set of experiments, doubling the amount of cells to 80 ml of 4×10⁵ cells/ml solution, increased the radioactivity of all the pseudopterosin compounds (PsA through D) to 818±89 DPM and the DPM of elisabethatriene was 525±26 DPM. The later optimization experiments included increasing the concentration of cells per ml, increasing the specific activity of the added ¹⁴C labeled sodium bicarbonate (NaH¹⁴CO₃) to 2 μCi/ml and increasing the time of incubation to 48 hours. The optimization increased the amount of radioactive pseudopterosin compounds by 66% to 2427±540 and the radioactivity of elisabethatriene increased to 4275±540 DPM.

In all these experiments, the pseudopterosin compounds were clearly found to be radioactive, thereby indicating the incorporation of NaH¹⁴CO₃ during the biosynthesis of the pseudopterosin compounds. This conversion of inorganic carbon into the pseudopterosin compounds indicate that photosynthesis is a source carbon for pseudopterosin production. In cells treated with darkness to inhibit photosynthetic incorporation of NaH¹⁴CO₃ the pseudopterosins were not radioactive, further proving the conclusion that these pseudopterosin compounds are not produced as a result of animal metabolism, but originate in the symbiont.

The diterpene cyclase produced and the intermediate, elisabethatriene, was radioactive, thereby indicating the incorporation of NaH¹⁴CO₃ during the biosynthesis of pseudopterosin compounds. Incorporation of NaH¹⁴CO₃ into putative intermediates of pseudopterosin biosynthesis indicates the assimilation of the label into the carbon backbone of the pseudopterosin compounds.

### Example 3

### Chemical Analysis of Extracts

About 100 grams of flash frozen P. elisabethae was ground in a blender with deionized water to release the Symbiodinium spp. from the coral host tissue. The resulting slurry was centrifuged at 7000 RPM for 10 minutes using a Sorvall RC-5 centrifuge at 4° C., and both the pellet and supernatant were examined under a light microscope for the presence of Symbiodinium spp. The pellet was rinsed with deionized water, and centrifuged repeatedly to remove host cellular material. The pellet was then placed on a discontinuous Percoll gradient of 80%, 60%, 40%, 20%, and 0% and centrifuged to collect the algae rich layers. Each layer was inspected using light microscopy, and layers containing high quantities of Symbiodinium spp. (80/40) were pooled. These combined layers were rinsed with deionized water and centrifuged an additional three times, and then subjected to additional Percoll gradients until the Symbiodinium spp. layers were at least about 95% free of contaminants. The algal pellet was then rinsed and centrifuged using deionized water five times. The pellet was frozen using liquid nitrogen and lyophilized to obtain a dry cell weight of 50 mg.

The dry cells were extracted with hexanes, and the resulting extract was partitioned between hexanes and methanol:water (9:1). The hexanes layer was analyzed by reverse phase HPLC using 100% methanol and a photodiode array detector against a known standard of elisabethatriene. The presence of elisabethatriene was confirmed by NMR analysis. NMR analysis of the HPLC peak showed features characteristic of elisabethatriene. For comparison, see Kerr, R. G. et al. (2000) Tetrahedron 56:9569-9574, which is herein incorporated by reference.

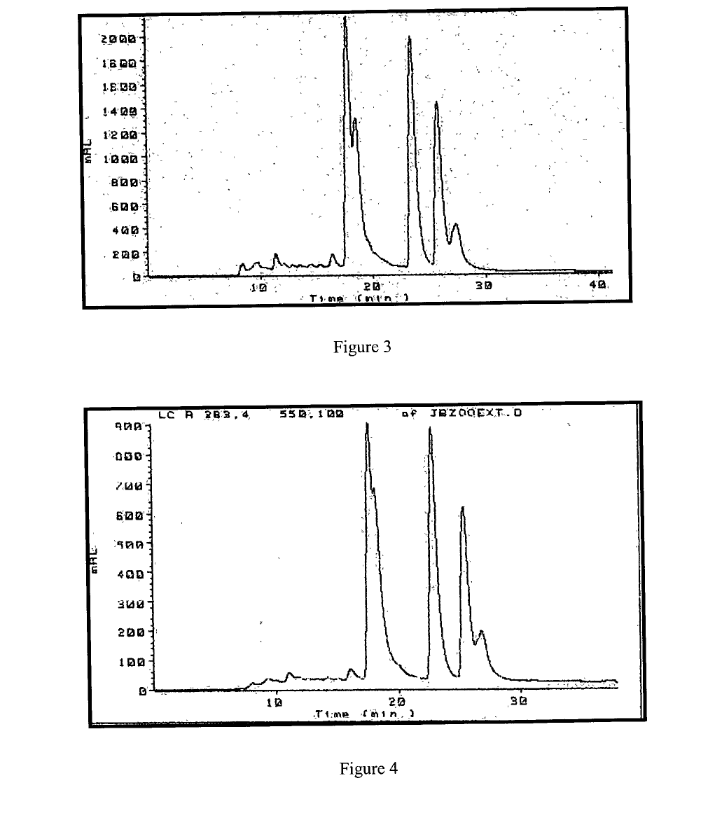

The remaining methanol:water fraction was adjusted to a 1:1 ratio and extracted with chloroform. The chloroform fraction was analyzed using normal phase HPLC and gradient elution from 60/40 hexanes:ethyl acetate to 100% ethyl acetate over 25 minutes, against known standards of Pseudopterosins A-D. FIG. 3 is a chromatogram of the chloroform fraction of a standard for Pseudopterosins A-D. FIG. 4 is a chromatogram of the chloroform fraction from Symbiodinium spp. which indicates that Pseudopterosins A-D may be isolated from Symbiodinium spp.

Pseudopterosins A-D were collected and a total pseudopterosin weight of 7.6 mg was obtained. Thus, the pseudopterosin yield of about 15% was obtained from the extracted, purified, dry Symbiodinium spp.. The coral/symbiont association typically yields an extract containing 7% pseudopterosins. Thus, extracts of purified Symbiodinium spp. provide a higher yield of pseudopterosin compounds as compared to coral extracts. The higher yield of pseudopterosin compounds from the algae extract provides higher pseudopterosin compound activity per gram of total extract.

### Example 4

### In Vitro Analysis with ³H Geranylgeranyl Pyrophosphate

Algal cells were separated from flash frozen coral collected from the Bahamas. The soft coral was blended in a Waring blander with filtered seawater and EDTA. The homogenate was filtered through 4 layers of cheesecloth. The filtrate was centrifuged at 1000 g to yield an algal pellet. The algal pellet was rinsed with filtered seawater and centrifuged 10 times. The algae was concentrated and placed on a Percoll step gradient of 80%/40%/20% Percoll dilutions in deionized water. The 80/40 layer and 40/20 layer were collected and rinsed with filtered seawater. The 40/20 layer was reapplied to the same step gradient and the 80/40 layer was collected. The 80/40 layer was combined and reapplied to the same step gradient. The 80/40 layer was recollected, rinsed with filtered seawater, and concentrated using centrifugation.

A cell free extract of the purified algal pellet was prepared by diluting the cells in 5 ml of 1 mM phosphate buffer, pH 7.7, with 5 mM of MgCl₂. Cells were disrupted in a French press. The cell free extract was incubated with 5 μCi ³H labeled geranylgeranyl pyrophosphate (GGPP) for 24 hrs. The reaction was quenched and extracted in chloroform. The crude extract was portioned between hexanes and (9:1) Methanol (MeOH)/water. The MeOH/water extract was run on straight phase HPLC on a gradient from 60% hexane/40% ethyl acetate to 100% ethyl acetate. Pseudopterosin A, B, C and D were collected from the HPLC and rerun again on the HPLC for further purification, the pseudopterosins were then collected every minute for radioisotope analysis on liquid scintillation counter.

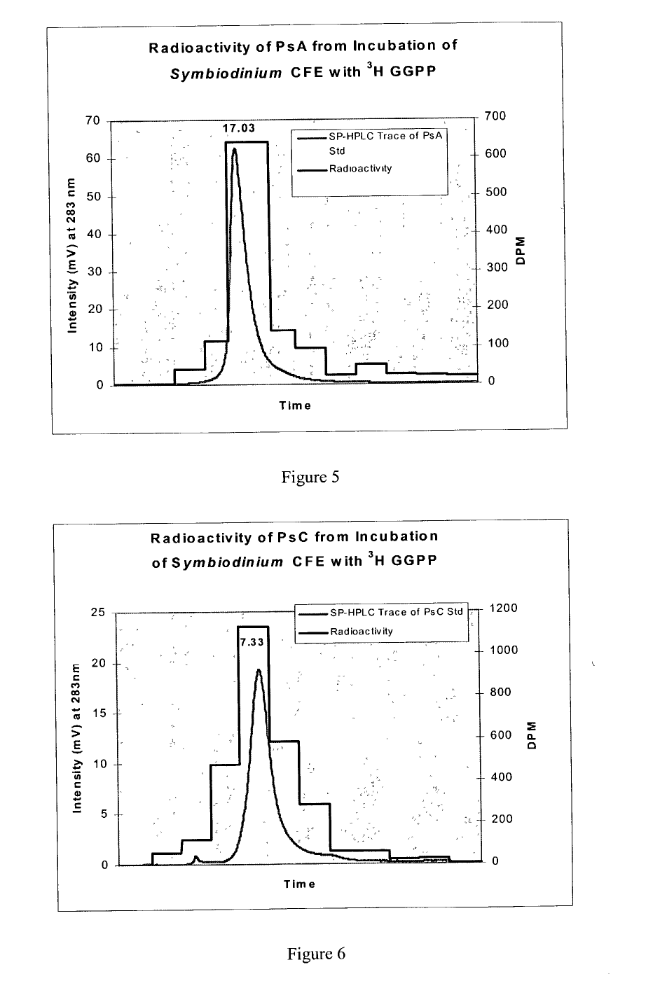

As shown in FIGS. 5 and 6, geranylgeranyl pyrophosphate is incorporated into PsA and PsC, thereby indicating the necessary biosynthetic machinery in Symbiodinium spp. for the synthesis of pseudopterosin compounds. The amounts of PsA and PsC were calculated as DPM/mg protein of the cell free extract was 1457 and 2568 for PsA and PsC, respectively.

Further optimization of the cell free system included changing the cell disruption buffer from phosphate to Tris buffer and adding the dispersing agent Triton X-100 to help solubilize geranylgeranyl pyrophosphate and optimize substrate micelle concentration. These experimental changes increased the radioactivity of all the pseudopterosin compounds (PsA through D) to 69,047 DPM. The pseudopterosin compounds were not radiolabeled when the cell free extract was boiled 1 hour prior to incubation.

### Example 5

### Cultivation of Symbiodinium spp. for Production of Pseudopterosin Compounds

Pseudopterosin compounds may be produced by cultivating Symbiodinium spp. Specifically, a culture of Symbiodinium spp. isolated from P. elisabethae is obtained by homogenizing P. elisabethae in a blender with filtered seawater and 10% EDTA. The mixture is filtered through about 4 to about 6 layers of cheesecloth into Erlenmeyer flask to remove coral skeletal parts. The algae cells are then diluted and rinsed through repeated centrifugations in fresh filtered seawater. This process in repeated about 10 times or until no coral tissue is visible under light micropscopy. The algae cells are further cleaned by application to a step Percoll gradient with 80% 40% and 20% Percoll in deionized water. The cells at the 80/40 interface are collected and reapplied to a new gradient for further clean up. The cells are checked for purity under light microscopy.

Clean algae cells are then cultured by growing in f/2 enriched seawater media purchased from Sigma-Aldrich (St. Loius, Mo.) in a 27° C. incubator with full spectrum lighting especially for plant growth under conditions which allow the production of pseudopterosin compounds. The pseudopterosin compounds are then purified from cell free extracts of the cultured algae cells by extraction in 100 ml of chloroform and purification on straight phase HPLC. A crude aqueous extract of the host coral (P. elisabethae) may be added to the growing cultures to stimulate production and extraction of the compounds into the culture medium.

### Example 6

### Pharmacological Evaluation of Pseudopterosin Compounds

Many pseudopterosin compounds have been found to be effective anti-inflammatory agents, anti-proliferative agents and analgesic agents for the use in treating mammals. The pharmacological activity of pseudopterosin compounds produced by or isolated from Symbiodinium spp. may be determined by the following assays:

A. Inhibition of PMA-Induced Inflammation (Edema) of the Mouse Ear

A test compound is topically applied in acetone to the inside pinnae of the ears of mice in a solution containing the edema-causing irritant, phorbol 12-myristate 13-acetate (PMA). PMA alone (2 μg/ear) or in combination with the test compound is applied to left ears (5 mice per treatment group) and acetone is applied to all right ears. After 3 hour 20 minute incubation, the mice are euthanized, the ears are removed, and bores are taken and weighed. Edema is measured by subtracting the weight of the right ear (acetone control) from the weight of the left ear (treated). Results are recorded as % decreases (inhibition) or % increase (potentiation) in edema relative to the PMA control group edema. Test compounds are screened at 50 or 25 μg/ear.

B. Myeloperoxidase Extraction and Assay

The neutrophil-specific marker, MPO, in ear biopsies from treated and untreated mouse ears is extracted and quantitated according to a modified method of Bradley. Samples from each group (10 mice per group) are pooled and homogenized in 2.0 ml of 80 mM sodium phosphate buffer (pH 5.4) containing 0.5% hexadecyltrimethylammonium bromide in a siliconized glass test tube for 1 min at 0° C. using a Brinkman Polytron. The polytron is washed with 1 ml of buffer, the wash is combined with homogenates and the mixture is centrifuged at 10,000×g at 4° C. for 30 min. Samples (10 μl) from each group are then assayed in a 96-well microtiter plate. The assay is initiated by adding 250 μl of o-dianisidine/phosphate reagent (0.28 mg of dianisidine added to 1 ml of 50 mM sodium phosphate comprising 0.0015% H₂O₂) to each well. After a thirty minute incubation at 37° C., the plates are read at 450 nm on a kinetic microplate reader such as Molecular Devices Vₘₐₓ kinetic microplate reader. Optical density values from drug-treated groups are compared with control groups to determine percent inhibition.

C. Fertilized Sea Urchin Egg Inhibition of Cleavage Assay for Anti-Proliferation

To determine whether a compound of the invention exhibits anti-proliferative activity, either cytostatic or cytotoxic, sea urchins are induced to spawn by injection of 0.5M KCl into the coelomic cavity. The test compound is added to a 1% slurry of eggs within 5 minutes following fertilization and incubated until the completion of the division in control slurry, about 90 to about 120 minutes. Inhibition is measured as the percent of undivided cells in the slurry at the end of this incubation. Compounds of the invention which are cytostatic may be used to block the progression of the cell cycle for studies in addition to treating diseases and disorders related to abnormal cell proliferation.

D. Phenylquinone Assay for Analgesia

To determine whether a pseudopterosin compound exhibits analgesic properties, a test compound is injected subcutaneously into mice. After 30 minutes, phenylquinone is injected intraperitoneally to cause pain as indicated by writhing. Absence of or a statistically significant decrease in writhing is considered evidence of analgesia. See Hendershot, L. C. and G. Forsaith, (1959) Pharmacol. Exp. Ther. 125:237, which is incorporated by reference.

E. Tetrahymena thermophila as a Pharmacological Model

As PsA and PsE are both active in reducing PMA-induced mouse ear edema when administered topically (ED₅₀=8 μg/ear and 38 μg/ear, respectively) and PsA inhibits prostaglandin E₂ and leukotriene C₄ production in zymosan-stimulated murine peritoneal macrophages (IC₅₀=4 μM and 1 μM, respectively), pseudopterosin compounds may mediate anti-inflammatory effects by inhibiting eicosanoid release from inflammatory cells in a concentration and dose-dependant manner. This signaling mechanism may be coupled to phagosome formation. The endogenous production of eicosanoids and their role in phagosome formation may be evaluated by using Tetrahymena as pharmacological probes and analyzing arachidonic acid products by conventional means.

In Tetrahymena thermophila, PsA and elisabethadione were potent inhibitors of phagocytic activity with IC₅₀ of 0.4 and 0.1 μM, respectively. A test compound may be assayed as follows. Log-phase Tetrahymena thermophila cultures are grown in 2% proteose peptone, 0.1% glucose at 25° C. A volume comprising 1×10⁷ cells are centrifuged at 450 g for 5 minutes. The pellet is washed and resuspended in 3.15 ml of 50 mM TRIS-HCl buffer at pH 7.7. The test compound is prepared at a desired concentration in 0.4 ml volumes. For control samples, 0.4 ml of buffer is used. 0.45 ml of diluted India ink (1:25, v:v) is added to each drug preparation. At t=0, the drug/ink mixture is added to the Tetrahymena cells, and at t=10 min., 500 ml samples of cell suspension are removed and fixed in 10% formaldehyde. At least 100 random cells from each sample are then examined in a light microscope. Phagocytic activity is assessed by calculating the ratio of cells with food vacuoles compared to the cells with no food vacuoles.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the specific embodiments as illustrated herein, but is only limited by the following claims.

What is claimed is:

1. A method of obtaining, isolating, purifying or preparing at least one pseudopterosin compound comprising obtaining, isolating, purifying or preparing the compound from a Symbiodinium spp. symbiont.

2. The method of claim 1, wherein the Symbiodinium spp. symbiont belongs to phylotype B1.

3. The method of claim 1, wherein the Symbiodinium spp. symbiont is obtained from a host.

4. The method of claim 3, wherein the host is Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Plexaura, Pocillopora, Pseudoterogorgia, Rhodactis, Stylophora, Tridacna, and Zoanthus.

5. The method of claim 3, wherein the host is Pseudopterogorgia.

6. The method of claim 5, wherein the host is P. elisabethae.

7. The method of claim 1, which further comprises culturing the Symbiodinium spp. symbiont.

8. The method of claim 1, wherein the pseudopterosin compound is selected from the group consisting of pseudopterosins, seco-pseudopterosins, diterpene aglycones, tricyclic diterpenes, and derivatives thereof.

9. The method of claim 1, wherein the pseudopterosin compound is a naturally occurring compound.

10. The method of claim 1, wherein the pseudopterosin compound is a synthetic compound.

11. The method of claim 1, wherein the pseudopterosin compound is Pseudopterosin A, Pseudopterosin B, Pseudopterosin C, Pseudopterosin D, Pseudopterosin E, Pseudopterosin F, Pseudopterosin G, Pseudopterosin H, Pseudopterosin I, Pseudopterosin J, Pseudopterosin K, Pseudopterosin L, Seco-Pseudopterosin A, Seco-Pseudopterosin B, Seco-Pseudopterosin C, Seco-Pseudopterosin D, Seco-Pseudopterosin E, or elisabethatriene.

12. The method of claim 1, wherein the pseudopterosin compound is Pseudopterosin A, Pseudopterosin B, Pseudopterosin C, Pseudopterosin D, or elisabethatriene.

13. The method of claim 1, which further comprises incubating the Symbiodinium spp. symbiont with NaHCO₃.

14. A pseudopterosin compound or pseudopterosin composition obtained by the method of claim 1.

15. The pseudopterosin compound of claim 14, wherein the pseudopterosin compound is a glycoside.

16. The pseudopterosin compound of claim 15, wherein the glycoside is modified.

17. The pseudopterosin compound of claim 14, wherein the pseudopterosin compound or pseudopterosin composition is substantially free of animal impurities.

18. The pseudopterosin compound of claim 14, wherein the pseudopterosin compound or pseudopterosin composition is of non-animal origin.

19. A pharmaceutical composition comprising at least one pseudopterosin compound or at least one pseudopterosin composition of claim 14 in a therapeutically effective amount and a pharmaceutically acceptable carrier.

20. A cosmetic composition comprising at least one pseudopterosin compound or at least one pseudopterosin composition of claim 14 in a therapeutically effective amount and a cosmetically acceptable carrier.

21. A method for treating, preventing, or inhibiting an infection, disease, or disorder related to an organism belonging to the kingdom Protista in a subject comprising administering to the subject a therapeutically effective amount of at least one pseudopterosin compound or at least one pseudopterosin composition.

22. The method of claim 21, wherein the pseudopterosin compound is selected from the group consisting of pseudopterosins, seco-pseudopterosins, diterpene aglycones, tricyclic diterpenes, and derivatives thereof.

23. The method of claim 21, wherein the pseudopterosin compound is a naturally occurring compound.

24. The method of claim 21, wherein the pseudopterosin compound is a synthetic compound.

25. The method of claim 21, wherein the compound is Pseudopterosin A, Pseudopterosin B, Pseudopterosin C, Pseudopterosin D, Pseudopterosin E, Pseudopterosin F, Pseudopterosin G, Pseudopterosin H, Pseudopterosin I, Pseudopterosin J, Pseudopterosin K, Pseudopterosin L, Seco-Pseudopterosin A, Seco-Pseudopterosin B, Seco-Pseudopterosin C, Seco-Pseudopterosin D, Seco-Pseudopterosin E, or elisabethatriene.

26. The method of claim 21, wherein the pseudopterosin compound is Pseudopterosin A, Pseudopterosin B, Pseudopterosin C, Pseudopterosin D, or elisabethatriene.

27. The method of claim 21, wherein the pseudopterosin compound is obtained, isolated, purified or prepared from an organism belonging to the genus Symbiodinium.

28. The method of claim 21, wherein the organism is a flagellate, a ciliate, an opalinidae, or a sporozoan.

29. The method of claim 21, wherein the organism is a plasmodium, a trypanosome, tetrahymenium, or a paramecium.

30. The method of claim 21, wherein the infection, disease, or disorder is malaria, Chagas' disease, African sleeping sickness, Leishmaniasis, giardiasis, or amebic dysentery.

31. The method of claim 21, wherein the organism is trichinosis, trypanosomiasis, leishmania, filariasis, or dracunculiasis.

32. A method of treating, preventing, or inhibiting a disease or disorder associated with inflammation, cell proliferation, pain, or a combination thereof in a subject comprising administering to the subject a therapeutically effective amount of at least one pseudopterosin compound or at least one pseudopterosin composition of claim 14.

33. An extract comprising at least one pseudopterosin compound in an amount that is greater than amounts obtained from coral extracts.

34. The extract of claim 33, wherein the extract is an algal extract.

35. The extract of claim 33, wherein the extract exhibits a greater pseudopterosin compound activity per gram of extract as compared to coral extracts.
